(19) United States
(12) Patent Application Publication
(10) Pub. No.: US 2008/0312649 A1
(43) Pub. Date: Dec. 18, 2008

(54) Illuminated instrument buttons

(76) Inventors: Paul Guerra, Boulder, CO (US); Dylan Hushka, Boulder, CO (US)

Correspondence Address: Tyco Healthcare Group LP, 60 MIDDLETOWN AVENUE, NORTH HAVEN, CT, 06473, US

(21) Appl. No.: 11/820,075

(22) Filed: Jun. 18, 2007

(51) Int. Cl. A61B 18/14

(52) U.S. Cl. 606/41

(57) ABSTRACT

An illuminated control surface is disclosed for use on a surgical instrument. The control surface may be disposed on a pushbutton switch located on a working head adapted for controlling the surgical tool. The light source may be an LED mounted remotely with respect to the illuminated control surface with light directed toward the control surface by a fiber optic strand. A translucent material may be selected for forming the control surface such that light may be directed through the material to illuminate the surface. Various colors and illumination patterns may be used to provide visual queues as to the status and operation of the instrument.

### BACKGROUND

1. Technical Field

The present disclosure relates generally to surgical instruments and more specifically to surgical instruments having illuminated control surfaces and related methods.

2. Background of Related Art

A surgeon will often need to cut tissue, occlude vessels or perform some other procedure at an operative site on or within a patient. Instruments developed to facilitate these processes typically include a surgical tool on the distal end, appropriately configured to manipulate the targeted tissue, and a working head on the proximal end which the surgeon can use to control the position and operation of the tool. In some cases, a surgical instrument will additionally include a remote console coupled to the tool through wires, hoses or other flexible apparatus allowing for the free movement of the working head and tool. These remote consoles may provide fluids, electrical energy or other inputs to the tool and operative site.

A conventional open surgical procedure involves a relatively large incision made in the body tissue in order to gain access the operative site. This practice exposes interior tissue to the open environment making it susceptible to infection and also requires a substantial portion of the body to heal after the surgery. An endoscopic or laparoscopic procedure, on the other hand, may reduce these difficulties by relying only on a small portal for access, which may be created by a puncture-like incision through the skin. A surgeon may insert an endoscope through the portal to view the operative site and determine how best to manipulate the other instruments. While it is not unusual for a surgeon to look directly into an endoscope through an ocular lens, it is more common for an endoscope to be associated with a camera system allowing the surgeon to view images on a video screen. In order to assist the surgeon in viewing these images, an endoscopic operating room may be darkened making it difficult to see the working head of an instrument.

Endoscopic surgery is possible due in part to the availability of instruments designed specifically for this purpose. Such an instrument typically has an elongated body such that it may be positioned through a narrow cannula of the type often used in endoscopic surgery to hold the portal open. The tool at the distal end is positioned within the body at the operative site, while the working head at the proximal end remains in the open environment to be handled by a surgeon. Because the operating room may be darkened, and because much of the surgeon's attention is directed to images of the operative site, the working head is best designed for intuitive control of the tool.

Some endoscopic instruments are designed to introduce electrical energy to an operative site in order to heat body tissue for various purposes. Electrosurgical forceps, for example, have been used to deliver a combination of electrical energy and mechanical clamping force to coagulate, cauterize and seal vessels. Generally, bipolar forceps grasp tissue between two poles disposed on pivotable jaws and apply an electrical current through the grasped tissue. A monopolar device, on the other hand, might deliver energy through a single pole where a remote return electrode is attached externally to the surgical subject. Bipolar energy is typically used for sealing vessels and vascular tissues while monopolar energy is typically used to coagulate or cauterize tissue. In either case, the current may be generated in a remote console and transmitted to the poles through a flexible cable. In some cases, a single procedure will require both types of energy (monopolar and bipolar), and some instruments have been adapted to selectively deliver both. An example of such an instrument is the endoscopic forceps described in U.S. patent application Ser. No. 11/540,335 by Patrick L. Dumbauld.

Control mechanisms are provided to activate the various functions of a surgical tool. For example, opposed handles may be provided on the working head of a forceps assembly, which a surgeon may manually pivot to close the jaws. Additionally, switches may be disposed on either the working head or a remote console to allow a surgeon to initiate the flow of electricity, select the intensity of energy provided, and select the appropriate mode. Preferably these switches will be located on the working head so that the surgeon will not need to divert attention from the operative site to engage them. Other features of an instrument may also allow for a more intuitive use of the tool.

### SUMMARY

The present disclosure describes an instrument equipped with a surgical tool on a distal end and equipped with a working head on the proximal end. An illuminated control surface is disposed on the working head to facilitate manipulation of the tool.

In a particular embodiment, the control surface is a pushbutton composed of a translucent material. Light from an LED light source in an interior cavity of the working head is directed through the button to illuminate the control surface. The color, intensity, or a pattern of illumination such as an intermittency (blinking) of the light emitted is adapted to provide information to a surgeon such as the location of the button in a darkened operating room, the function of the button, and an indication as to the appropriate use of the button. The light may be directed through a light pipe or fiber optic strand from a light source remotely located with respect to the button. Power for the light source may be provided by a battery within the instrument, or alternatively, power may be delivered by a remote console such as an electrosurgical generator.

A method is also described for controlling a surgical tool. The method involves providing a surgical tool for manipulating tissue, a control surface to assist an operator in controlling the tool, and a light source adapted to illuminate the control surface. The operator may then identify a function associated with the control surface by the illumination of the control surface and manipulate the control surface to perform the function with the tool. The function may be to provide electrosurgical energy to the tool and such a function may be identified through illumination with a distinguishing color.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1 is a top perspective view of an illustrative embodiment of the disclosure including an endoscopic forceps assembly with two illuminated buttons;

FIG. 2 is an enlarged perspective view of the internal components of the forceps of FIG. 1 showing the two illuminated buttons, a circuit board, and a cable;

FIG. 3 is top perspective view, with parts separated, of a control system for the forceps of FIG. 1 showing an electrosurgical generator connected to the forceps by a flexible cable; and

FIG. 4 is an enlarged partial side view of an illuminated button connected to the circuit board of FIG. 3.

### DETAILED DESCRIPTION

The present disclosure contemplates the introduction into a person's body of all types of surgical instruments including clip appliers, graspers, dissectors, retractors, staplers, laser fibers, photographic devices, endoscopes and laparoscopes, tubes, and the like. All such objects are referred to herein generally as “instruments.” In the drawings and in the description that follows, the term “proximal,” as is traditional, will refer to the direction toward the operator or a relative position on the surgical device or instrument that is closer to the operator, while the term “distal” will refer to the direction away from the operator or relative position of the instrument that is further from the operator.

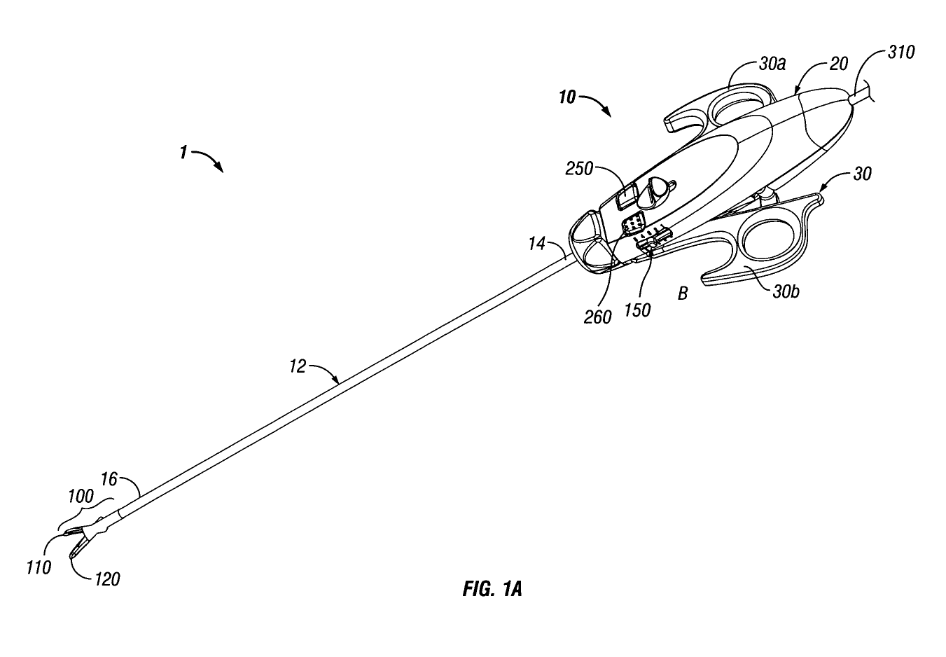

Referring initially to FIG. 1, a surgical instrument is depicted having two illuminated buttons 250, 260. The instrument depicted is a relatively complex environment for use with the present disclosure and takes the form of an inline endoscopic combination monopolar and bipolar forceps assembly 1, which may be used to electrosurgically treat tissue. Relevant components include end effector assembly 100, shaft 12 having a distal end 16 and a proximal end 14, working head 10 and cable 310. Working head 10 includes housing 20, intensity control 150, two illuminated buttons 250 and 260 and handle assembly 30 which itself includes handles 30a and 30b.

End effector assembly 100 is configured to be positioned within body tissue to manipulate the tissue by clamping, electrosurgically energizing, cutting and/or otherwise contacting the tissue. At least one of the jaws 110, 120 on the end effector may be adapted to deliver electrosurgical energy in a monopolar fashion to the surrounding tissue, and both jaws 110, 120 in combination may be adapted to deliver electrosurgical energy in a bipolar fashion. The jaws 110, 120 are further adapted to move between an open position, as shown in FIG. 1, where the distal-most ends are substantially spaced and a closed position where they are closer together.

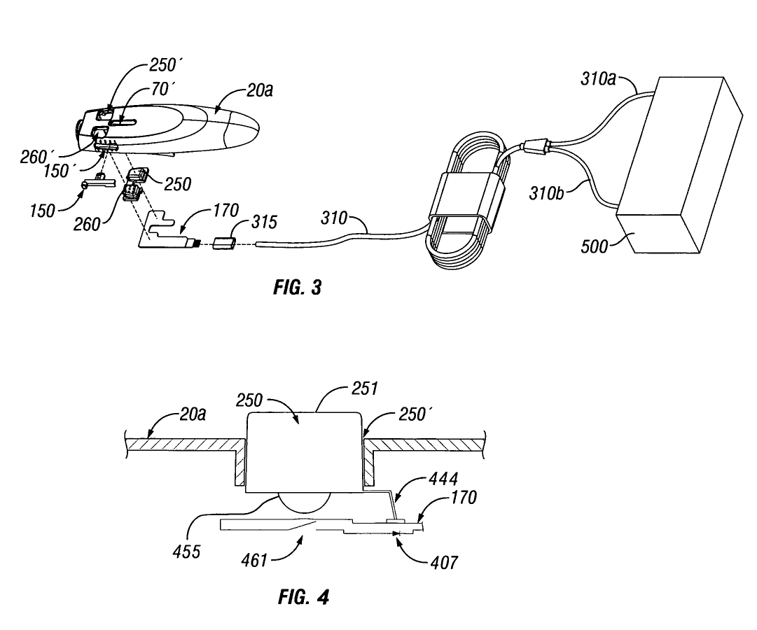

Elongated shaft 12 couples end effector 100 to working head 10 and is narrow such that it may be inserted through a cannula for use in endoscopic procedures. Handle assembly 30 includes two moveable handles 30a, and 30b disposed on opposite sides of housing 20. Handles 30a and 30b are moveable relative to one another to activate end effector assembly 100 and move the jaws 110, 120 between their open and closed positions. Housing 20 is sized appropriately to allow handles 30a, 30b to be grasped and operated by a single hand. Cable 310 extends from the proximal end of housing 20 and serves to generally transfer information and energy between the forceps assembly 1 and a remote generator 500 (depicted in FIG. 3). Intensity control 150 is coupled to cable 310 and end effector 100 such that the operator may select the intensity of energy delivered though the cable into the jaws of the end effector by sliding intensity control 150 in a proximal or distal direction.

Finally, protruding through housing 20 are two illuminated buttons 250, 260. Button 250, when depressed, causes the delivery of energy to the end effector in a bipolar fashion, while depressing button 260 causes energy to be delivered in a monopolar fashion. Button 260 includes an array of raised protuberances on a top surface to provide a visual and tactile queue to distinguish it from button 250. Alternatively, a single protrusion may suffice to distinguish the buttons 250, 260. A visual queue may also be provided through the illumination of the buttons, as discussed below.

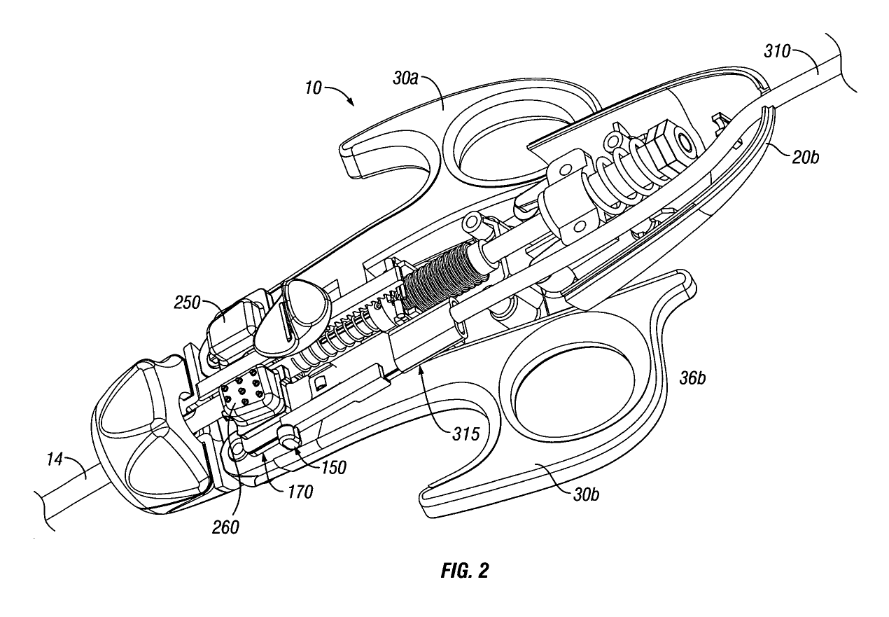

Referring now to FIG. 2, working head 10 is depicted with a top portion of housing 20 removed to show the inner components. Lower housing 20b extends across the underside of the working head 10 and permits entry of cable 310. Cable 310 is routed within working head 10 to overmold portion 315 where at least some of the individual conductors terminate and couple to circuit board 170. Intensity control 150 and buttons 250, 260 are also coupled to circuit board 170 and, therefore, these controls are in electrical communication with cable 310. Circuit board 170 is configured to receive inputs from the controls 150, 250, 260 and communicate electrical signals through cable 310 to generator 500 (shown in FIG. 3) as to the type of electrosurgical energy to be delivered to end effector 100.

FIG. 3 depicts the control system of the forceps assembly 1 including electrosurgical generator 500. Generator 500 is a remote source of both bipolar and monopolar electrosurgical energy coupled to cable 310 by leads 310a, 310b. Generator 500 is envisioned as a stationary component that may remain in place as forceps assembly 1 is maneuvered into position and used to perform the desired procedure. Generator 500 may include controls such as a power switch or safety mechanisms to limit the power levels delivered. Because of its remote location, controls disposed on generator 500 are preferably limited to those used only at the initial setup or final stages of a surgical procedure. Controls frequently accessed during the procedure may be more conveniently located on the working head 10 so the surgeon will not need to divert attention from the procedure to access them.

Cable 310 is shown wound into a bundle indicating that it may have a sufficient length to allow the surgeon some freedom of motion. At least some of the conductors of cable 310 lead into to the overmold portion 315 for connection with circuit board 170. Other conductors may continue on to end effector 100. Buttons 250, 260 are configured to seat within respective apertures 250′ and 260′ of upper housing 20a when assembled. Likewise, intensity control 150 is configured to slide within slot 150′ such that a portion of the intensity control 150 protrudes from upper housing 20a to modify the intensity of the electricity provided. The controls 150, 250, 260 are configured to effect changes in the circuitry found in circuit board 170. Electrical signals are then communicated through cable 310 to generator 500, which processes the signals to determine the appropriate type and level of energy to transmit to jaw members 110, 120.

FIG. 4 depicts a side cross sectional view of pushbutton 250 seated in aperture 250′ of upper housing 20a. An upper control surface 251 protrudes to an exterior side of housing 20a and is adapted to be displaced by a finger. A button plunger 455 is disposed on the underside of button 250 and is adapted for activating a tactile switch 461 coupled to a control circuit on circuit board 170 when control surface 251 is displaced. The control circuit is adapted to cause the instrument to perform its desired function, in this case for example, to initiate or cease the delivery of bipolar energy to jaws 110, 120. Light emitting diode (LED) 407 is disposed on circuit board 170 at a remote location relative to pushbutton 250. Light pipe 444 provides an optical path for the transmission of light emitted from LED 407 to pushbutton 250. Pushbutton 250 is formed at least partially from a translucent material, such that light entering from light pipe 444 will illuminate at least a portion of control surface 251.

Light pipe 444 may be any elongated transparent medium capable of transmitting light from LED 407 to pushbutton 250. A mechanical connection of the light pipe 444 to either LED 407 or pushbutton 250 is not necessary as long as optical communication is established. Any suitable optical and/or mechanical connection may accommodate the motion of pushbutton 250. A flexible fiber optic strand may be used as light pipe 444 and may be especially useful for transmitting light over relatively long distances, for example, from an LED light source on a remote circuit board not otherwise connected to the illuminated control. Suitable light sources other than LEDs may also be included. A traditional lamp mounted on a cable such that it is isolated from any circuit board may suffice. Power for the light source may be provided by a battery housed within the working head, or in a remote console, such as generator 500.

Also, it is contemplated that a control surface may be illuminated directly by a light source. For example, an LED may be positioned in the vicinity of a button such that at least a portion of the light emitted from the LED is directed directly through the control surface. In this way, a backlit button could be provided without the need for a light pipe.

The illumination of button 250 may serve at least one of several suitable purposes. First, the illumination may assist a surgeon in locating the button, for example, in a darkened endoscopic operating room. In this case, the light source may be independent of any control circuitry coupled to button 250. Button 250 may be adapted to remain constantly illuminated as long as LED 407 is powered, regardless of whether or not button 250 has been depressed. Secondly, the function of button 250 may be indicated through illumination. A distinguishing color, such as purple, may be used to indicate that button 250 activates the instrument's bipolar mode. This is especially useful when a similar button 260 is illuminated with a second distinguishing color, white for example, to indicate that button 260 activates the instrument's monopolar mode.

Methods of achieving illumination of distinguishing colors are well known in the art. For example, LEDs are commercially available in a wide variety of colors which may be appropriate for use in the present application. Alternatively, illumination of distinguishing colors may be achieved, for example, by the application of paint or ink to an appropriate surface in the light path. An appropriate surface may include control surface 251. Also, the color of the translucent material selected for button 250 may provide the color of the illumination. Thirdly, the illumination control surface 251 may be used to provide direction as to the use of button 250. For example, LED 407 may be coupled through the control circuitry to contact switch 461 such that it emits light only when the contacts on switch 461 are closed. In this way, an illumination of control surface 251 could indicate to a surgeon that the bipolar mode of the instrument had been selected and was currently active. Additionally, the illumination of control surface 251 could provide a warning to the surgeon. For example, a warning may be provided to prevent accidental activation of a particular mode of the instrument. It may be dangerous to activate the bipolar mode of the instrument when the jaws 110, 120 are situated in the open, spaced apart position. Safety control circuitry adapted to recognize the status of jaws 110, 120 could be adapted to cause LED 407 to emit light intermittently when jaws 110, 120 are situated such that it is unsafe to depress button 250. A flashing control surface might also direct the surgeon that the instrument has entered a lockout mode where depressing button 250 is ineffective. Alternatively, or in conjunction with a blinking button, a distinguishing color may be employed to indicate a condition satisfactory for depressing the button has been achieved.

Control surfaces other than those on buttons 250, 260 may also be illuminated. For example, intensity control 150 is associated with makings on upper housing 20 representing numerals 1 through 5. These markings are intended to provide a surgeon with a visual queue as to the effect of sliding intensity control 150 in one direction or the other. However, in a darkened operating room, these markings loose some of their effectiveness. A surgeon intending to lower the intensity of electricity delivered could easily slide intensity control 150 in the wrong direction and harm the patient. To help prevent this, the numerals themselves may be illuminated, or possibly a distinguishing color could be used at each end of aperture 150′ to provide a visual queue, for example, red at the distal end to represent higher intensity and blue at the proximal end to represent lower intensity. Such a visual queue might also be provided by varying the intensity of the light emitted from an aperture or surface. For example, the intensity of light emitted may be directly related to the intensity of the electrosurgical energy provided such that an increase in power delivered to the end effector 100 corresponds with an increase in LED power. Any knob, dial, handle, switch or other control mechanism, and any surfaces related to these control mechanisms, may be improved through illumination.

Further, control surfaces mounted on a remote console may be improved through illumination. Due to their remote location, it may be difficult for a surgeon to ascertain current settings or other information available from the control surfaces from a distance. Illuminating these surfaces may eliminate the need for a surgeon to redirect an external light or walk to the console to assess the information.

Although the foregoing disclosure has been described in some detail by way of illustration and example, for purposes of clarity or understanding, certain changes and modifications may be practiced within the scope of the appended claims.

What is claimed is:

1. An instrument for performing a surgical procedure, comprising:
a surgical tool adapted to manipulate tissue;
at least one control surface at least partially formed from a translucent material adapted to assist an operator in controlling the tool;
at least one light source mounted relative to the control surface adapted to transmit light through the translucent material to illuminate the control surface.

2. The instrument according to claim 1 wherein the control surface is disposed on a remote console adapted to provide electrical energy to the tool.

3. An instrument for performing a surgical procedure, comprising:
a surgical tool adapted to manipulate tissue;
a working head configured to position the surgical tool relative to the tissue, the working head including a housing;
at least one control surface coupled to the housing and adapted to assist an operator in controlling the tool; and
at least one light source coupled to the housing and adapted to illuminate the control surface.

4. The instrument according to claim 3 wherein the at least one control surface is at least partially formed from a translucent material.

5. The instrument according to claim 4 further comprising a light pipe adapted to direct light into the translucent material from the at least one light source.

6. The instrument according to claim 5 wherein the light pipe comprises a fiber optic strand.

7. The instrument according to claim 3 wherein the at least one light source includes an LED mounted on a circuit board housed within the working head.

8. The instrument according to claim 7 wherein a control mechanism associated with the control surface is coupled to the circuit board, and a control algorithm associated with the circuit board is adapted to produce a visible change in light emitted from the LED upon manipulation of the control mechanism.

9. The instrument according to claim 3 further comprising a plurality of control surfaces coupled to the housing, a respective one of the control surfaces configured to illuminate with a distinguishing color to identify it from another one of the control surfaces.

10. The instrument according to claim 3 further comprising control circuitry adapted to recognize a status of the instrument and communicate the status through a corresponding pattern of illumination of the control surface.

11. The instrument according to claim 10 wherein the pattern of illumination includes intermittent illumination of the control surface.

12. The instrument according to claim 3 wherein the at least one light source is powered by a remote console coupled to the working head.

13. The instrument according to claim 3 further including an elongated body adapting the instrument for use in an endoscopic procedure.

14. The instrument according to claim 3 further including an end effector disposed distally with respect to the working head, the end effector adapted to deliver electrosurgical energy to tissue.

15. A method for controlling a surgical tool, comprising the steps of:
providing a surgical tool adapted to manipulate tissue, the surgical tool including at least one control surface and at least one light source mounted relative to the control surface;
illuminating the control surface via the at least one light source to identify a function of the surgical tool; and
manipulating the illuminated control surface to perform the function.

16. The method according to claim 15 wherein the function associated with the control surface comprises delivering electrosurgical energy to the surgical tool.

17. The method according to claim 15 wherein the illuminating step comprises illuminating the control surface with a distinguishing color.

18. The method according to claim 15 further comprising communicating a status of the surgical instrument through a pattern of illumination of the control surface.
